(19) United States
(12) Patent Application Publication
(10) Pub. No.: US 2026/0063549 A1
(43) Pub. Date: Mar. 5, 2026

(54) FABRIC LIGHT TRANSMITTANCE TESTING APPARATUS

(71) Applicant: Quanzhou Normal University, Quanzhou (CN)

(72) Inventors: Lina CUI, Quanzhou (CN); Canyi HUANG, Quanzhou (CN); Pinghui WU, Quanzhou (CN)

(21) Appl. No.: 19/318,324

(22) Filed: Sep. 3, 2025

(30) Foreign Application Priority Data
Sep. 5, 2024 (CN) 202411241158.8

(51) Int. Cl. G01N 21/59; G01N 33/36

(52) U.S. Cl. CPC G01N 21/59; G01N 33/367

(57) ABSTRACT

A fabric light transmittance testing device comprises a supporting frame, a middle portion of an inner side of which is fixedly connected with a platform plate, a top surface of the supporting frame is fixedly connected with a top fixing plate having a top surface fixedly connected with a hydraulic rod, a bottom surface of the platform plate is fixedly connected with a light transmission testing mechanism, a cloth fastening mechanism is arranged above the light transmission testing mechanism, one side of the top fixing plate is provided with an unwinding control mechanism, four vertical sliding rods slidably penetrate through an upper surface of the top fixing plate, a bottom surface of the vertical sliding rod is fixedly connected with a detection bottom plate, a top surface of the platform plate is fixedly connected with a slope plate, and the light transmission testing mechanism comprises a side fixing plate.

### CROSS REFERENCE TO RELATED APPLICATIONS

This application claims foreign priority of Chinese Patent Application No. 202411241158.8, filed on September 5, 2024 in the China National Intellectual Property Administration, the disclosures of all of which are hereby incorporated by reference.

### TECHNICAL FIELD

The present invention relates to the technical field of fabric testing, and is particularly a fabric light transmittance testing device.

### BACKGROUND OF THE PRESENT INVENTION

Fabric light transmittance testing is usually intended to evaluate a fabric light transmittance performance, especially in the design and selection of curtains and sunshade fabrics, or other occasions where light transmittance needs to be considered, and the accuracy and repeatability of light transmittance testing are very important for fabric design and function selection.

The patent with the publication number CN108956548B discloses a fabric light transmittance testing device for garment production, which can facilitate cloth transmission, reduce local missing detection and improve use reliability; and can facilitate light source control, and make a detected part of cloth and a detector on the same horizontal plane, thereby reducing an influence on detection and improving the practicability; and the device comprises a detector; further comprises a supporting table, a group of supporting legs, a group of upper fixing plates, a group of lower fixing plates, a group of supporting frames, a group of upper fixing springs, a group of lower fixing springs, a group of upper clamping plates, a group of lower clamping plates, an upper rolling shaft, a lower rolling shaft, a group of upper fixing shafts and a group of lower fixing shafts; and further comprises a light source tank, a lamp holder, a group of lamp tubes, a group of upper connecting plates, a group of lower connecting plates, a group of upper positioning plates, a group of lower positioning plates, a group of upper limiting rollers, a group of lower limiting rollers, a group of upper limiting shafts, a group of lower limiting shafts, a supporting plate, an adjusting screw rod and a rotating plate. However, the patent has the problem that a structural shadow blocks light rays projected on the fabric, which affects a light transmittance. Therefore, it is necessary to design a fabric light transmittance testing device.

### SUMMARY OF THE PRESENT INVENTION

The present invention aims to provide a fabric light transmittance testing device, so as to solve the problem in the background above.

In order to solve the above technical problem, the present invention provides the following technical solution: a fabric light transmittance testing device comprises a supporting frame, wherein a middle portion of an inner side of the supporting frame is connected with a platform plate, a top surface of the supporting frame is fixedly connected with a top fixing plate, a top surface of the top fixing plate is fixedly connected with a hydraulic rod, a bottom surface of the platform plate is fixedly connected with a light transmission testing mechanism, a cloth fastening mechanism is arranged above the light transmission testing mechanism, one side of the top fixing plate is provided with an unwinding control mechanism, four vertical sliding rods slidably penetrate through an upper surface of the top fixing plate, a bottom surface of the vertical sliding rod is fixedly connected with a detection bottom plate, and a top surface of the platform plate is fixedly connected with a slope plate; the light transmission testing mechanism comprises a side fixing plate, the side fixing plate is fixedly connected to a rear side surface of the detection bottom plate, one side of the side fixing plate far away from the detection bottom plate is fixedly connected with a vertical-groove transverse plate, one side of the vertical-groove transverse plate far away from the side fixing plate is hinged with a double hinge rod, one end of the double hinge rod far away from the vertical-groove transverse plate is hinged with a single hinge rod, a hinged part between the double hinge rod and the single hinge rod is slidably connected with a guide groove rod, one end of the single hinge rod far away from the double hinge rod is connected with a lifting frame, two sides of the lifting frame are both slidably connected with a frame guide rod, an inner side of the lifting frame far away from the frame guide rod is fixedly connected with a thin rod, one end of the thin rod far away from the lifting frame is fixedly connected with a guide frame rod, an outer side of the guide frame rod is slidably connected with an inclined groove plate, a side surface of the inclined groove plate is fixedly connected with a reflective plate, a bottom surface of the lifting frame is fixedly connected with a bending rod, and one end of the bending rod far away from the lifting frame is fixedly connected with a light source plate; a bottom surface of the detection bottom plate is provided with a photosensitive sensor, the guide groove rod is fixedly connected with a rear side surface of the slope plate, the frame guide rod is fixedly connected with a bottom surface of the platform plate, and the reflective plate is hinged with the bottom surface of the platform plate. The hydraulic rod extends downwardly to drive the detection bottom plate to move downwardly, the detection bottom plate drives the vertical-groove transverse plate on a side surface of the side fixing plate to move together, and a joint between the double hinge rod and the single hinge rod for connecting the vertical-groove transverse plate slides along the guide groove rod. At this time, the lifting frame connected with the single hinge rod moves upwardly along the frame guide rod to approach the bottom surface of the platform plate, and the bending rod connected with the lifting frame pushes the light source plate to approach a bottom portion of cloth. A light source emitted by the light source plate passes through the cloth to reach the bottom surface of the detection bottom plate, and the photosensitive sensor on the bottom surface of the detection bottom plate receives a light source signal and transmits the light source signal to a terminal. The terminal analyzes a light signal intensity to judge a light transmission effect of the cloth, so as to achieve the function of testing the light transmittance of the cloth. Meanwhile, the upward movement of the lifting frame may drive the connected thin rod and guide frame rod to move upwardly, and the guide frame rod moves upwardly and slides along the inclined groove plate, so that the inclined groove plate drives the connected reflective plate to deflect around a joint with the bottom surface of the platform plate, and the deflected reflective plate reflects the light rays emitted by the light source plate.

According to the above technical solution, the cloth fastening mechanism comprises a vertical sliding plate, the vertical sliding plate is slidably connected to an upper surface of the vertical-groove transverse plate, the upper surface of the vertical-groove transverse plate slidably penetrates through two elastic rods, a bottom surface of the elastic rod is fixedly connected with an elastic rod fixing block, and one side of the elastic rod fixing block oriented to the vertical sliding plate is fixedly connected with a lower roller frame. Two upper roller frames are arranged above the lower roller frames, side surfaces of the upper roller frames and the lower roller frames are all rotatably connected with two cloth pressing roller shafts, a plurality of short rods are distributed on an outer surface of the cloth pressing roller shaft in an array, and one end of the short rod far away from the cloth pressing roller shaft is fixedly connected with a rubber arc strip. An outer side surface of the elastic rod is provided with a spring, two ends of the spring are respectively fixedly connected with the vertical-groove transverse plate and the elastic rod fixing block, the lower roller frame is fixedly connected with the vertical sliding plate, the vertical sliding plate penetrates from a top surface to a bottom surface of the vertical-groove transverse plate, and the upper roller frame is slidably connected with the side fixing plate. A spring leaf is arranged inside the side fixing plate, two ends of the spring leaf are respectively fixedly connected with the side fixing plate and the upper roller frame, the short rod is slidably connected with the cloth pressing roller shaft, one end of the short rod close to the cloth pressing roller shaft is provided with a spring, and two ends of the spring are respectively fixedly connected with the cloth pressing roller shaft and the short rod. Before the hydraulic rod extends downwardly, there is a gap between the vertical-groove transverse plate and the elastic rod fixing block formed through an elastic force of the spring outside the elastic rod, there is a maximum distance between the lower roller frame and the upper roller frame at this time, and there is a gap between the cloth pressing roller shaft of the lower roller frame and the cloth pressing roller shaft of the upper roller frame. At this time, the cloth is put in from the lower roller frame on one side without blocking, and the cloth passes through a space between the cloth pressing roller shaft of the lower roller frame and the cloth pressing roller shaft of the upper roller frame, which facilitates putting the cloth in a designated position before testing, and saves time for the cloth to pass through. When the hydraulic rod starts to extend downwardly, the vertical-groove transverse plate downwardly drives the elastic rod and the elastic rod fixing block to downwardly approach the slope plate, the lower roller frame connected with the elastic rod fixing block approaches the slope plate to make the cloth pressing roller shaft on the lower roller frame contact with the slope plate through the rubber arc strip, and the side fixing plate continues to move downwardly at this time, so that the upper roller frame connected with the side fixing plate contacts with the cloth through the rubber arc strip on the cloth pressing roller shaft and squeezes the cloth pressing roller shaft on the lower roller frame. The two cloth pressing roller shafts are mutually contacted and pressed through the adaptive arc plates, so that the rubber arc strips push the short rods to retract into the cloth pressing roller shafts to squeeze the springs, and at this time, the pressed rubber arc strips may contact with the cloth together with other rubber arc strips, thereby increasing a contact area of the cloth and fixing the cloth. In addition, the cloth pressing roller shaft on the lower roller frame may rotate around the cloth pressing roller shaft on the lower roller frame, so that the lower roller frames on two sides of the side fixing plate pull the springs and move away from each other.

According to the above technical solution, the unwinding control mechanism comprises a T-shaped connecting rod, the T-shaped connecting rod is arranged on one side of the top fixing plate, one end of the T-shaped connecting rod far away from the vertical sliding rod is slidably connected with a pressing vertical rod, a bottom end of the pressing vertical rod is fixedly connected with a stressed plate, a top surface of the stressed plate is fixedly connected with two vertically pressing connecting rods, a top end of the vertically pressing connecting rod is slidably connected with a fixing transverse plate, one side of the stressed plate far away from the pressing vertical rod is fixedly connected with a rolling arc plate, one end of the rolling arc plate far away from the stressed plate is fixedly connected with an arc plate connecting rod, one end of the arc plate connecting rod far away from the rolling arc plate is slidably connected with an adaptive arc plate, a cloth winding roller is arranged below the adaptive arc plate, and a side surface of the cloth winding roller is rotatably connected with a bracket. The T-shaped connecting rod is fixedly connected with two vertical sliding rods, the outer side of the pressing vertical rod is provided with the spring, and two ends of the spring are respectively fixedly connected with the T-shaped connecting rod and the stressed plate. The fixing transverse plate is fixedly connected with the supporting frame, the outer side of the arc plate connecting rod is provided with the spring, and two ends of the spring are respectively fixedly connected with the rolling arc plate and the adaptive arc plate. The supporting frame is fixedly connected with the bracket, and when the vertical sliding rod moves along with the downward extension of the hydraulic rod, the movement of the vertical sliding rod may drive the connected T-shaped connecting rod to move, and the T-shaped connecting rod moves downwardly to push the pressing vertical rod and the stressed plate to move. At this time, the rolling arc plate and the adaptive arc plate do not contact with the cloth outside the cloth winding roller, and when the T-shaped connecting rod moves downwardly, the spring on the outer side the pressing vertical rod is not pressed and moves downwardly together with the stressed plate. When the rolling arc plate and the adaptive arc plate contact with the cloth winding roller, the stressed plate connected with the rolling arc plate is subjected to a reverse action, so that the stressed plate approaches the T-shaped connecting rod and squeezes the spring on the outer side of the pressing vertical rod, and the cloth outside the cloth winding roller is pressed through the stressed plate, thereby preventing the cloth wound outside the cloth winding roller from being loose and offset to affect cloth detection. Meanwhile, with the unwinding of the cloth outside the cloth winding roller, a diameter of the cloth wound outside the cloth winding roller will be gradually reduced, and at this time, the rolling arc plate and the adaptive arc plate covered above the cloth winding roller may make the rolling arc plate and the adaptive arc plate approach each other under an elastic force of the spring outside the arc plate connecting rod.

Compared with the prior art, the present invention has the beneficial effects as follows:

according to the present invention, the side fixing plate, the vertical-groove transverse plate, the double hinge rod, the guide groove rod, the single hinge rod, the lifting frame, the frame guide rod, the bending rod, the light source plate, the thin rod, the guide frame rod and the inclined groove plate are provided, and the terminal analyzes the light signal intensity to judge the light transmission effect of the cloth, thereby achieving the function of testing the light transmittance of the cloth; meanwhile, the upward movement of the lifting frame may drive the connected thin rod and the guide frame rod to move upwardly, and the guide frame rod moves upwardly and slides along the inclined groove plate, so that the inclined groove plate drives the connected reflective plate to deflect around the joint with the bottom surface of the platform plate, and the deflected reflective plate will reflect the light rays emitted by the light source plate, thereby improving the uniformity of light rays projected on the cloth, increasing a light irradiation range, and avoiding a structural shadow from blocking the light rays to affect the light transmittance of the cloth;

according to the present invention, the vertical sliding plate, the elastic rod, the elastic rod fixing block, the lower roller frame, the upper roller frame, the cloth pressing roller shaft, the short rod and the rubber arc strip are provided, so that the rubber arc strips are pressed to push the short rods to retract into the cloth pressing roller shafts to squeeze the springs, and at this time, the pressed rubber arc strips may contact with the cloth together with other rubber arc strips, thereby increasing a contact area of the cloth and fixing the cloth; and in addition, the cloth pressing roller shaft on the lower roller frame may rotate around the cloth pressing roller shaft on the lower roller frame, so that the lower roller frames on two sides of the side fixing plate pull the springs and move away from each other, thereby fastening the cloth contacting with the cloth pressing roller shaft, and preventing the cloth from being loose and wrinkled to affect the accuracy of light transmission testing; and

according to the present invention, the T-shaped connecting rod, the pressing vertical rod, the stressed plate, the vertically pressing connecting rod, the fixing transverse plate, the rolling arc plate, the arc plate connecting rod, the adaptive arc plate, the bracket and the cloth winding roller are provided, and the stressed plate presses the cloth outside the cloth winding roller, thereby preventing the cloth wound outside the cloth winding roller from being loose and offset to affect cloth detection; and meanwhile, the rolling arc plate and the adaptive arc plate covered above the cloth winding roller may make the rolling arc plate and the adaptive arc plate approach each other under the elastic force of the spring outside the arc plate connecting rod, thereby improving a binding effect of the cloth outside the cloth winding roller, and improving the applicability to cloth rolls with different diameters outside the cloth winding roller.

### DESCRIPTION OF THE DRAWINGS

The drawings are provided to further understand the present invention and constitute a part of the specification. The drawings are used together with embodiments of the present invention to explain the present invention, but are not intended to limit the present invention.

In the drawings:

FIG. 1 is a schematic diagram of a front stereoscopic structure of an entirety of the present invention;

FIG. 2 is a schematic diagram of a rear stereoscopic structure of the present invention;

FIG. 3 is a schematic structural diagram of a light transmission testing mechanism of the present invention;

FIG. 4 is a schematic structural diagram of a bottom surface of a platform plate of the present invention;

FIG. 5 is a schematic diagram of an enlarged structure of a part A in FIG. 4;

FIG. 6 is a schematic structural diagram of a cloth fastening mechanism of the present invention; and

FIG. 7 is a schematic structural diagram of an unwinding control mechanism of the present invention.

In the drawings: 1 refers to supporting frame; 2 refers to platform plate; 3 refers to top fixing plate; 4 refers to hydraulic rod; 5 refers to light transmission testing mechanism; 51 refers to side fixing plate; 52 refers to vertical-groove transverse plate; 53 refers to double hinge rod; 54 refers to guide groove rod; 55 refers to single hinge rod; 56 refers to lifting frame; 57 refers to frame guide rod; 58 refers to bending rod; 59 refers to light source plate; 510 refers to thin rod; 511 refers to guide frame rod; 512 refers to inclined groove plate; 513 refers to reflective plate; 6 refers to cloth fastening mechanism; 61 refers to vertical sliding plate; 62 refers to elastic rod; 63 refers to elastic rod fixing block; 64 refers to lower roller frame; 65 refers to upper roller frame; 66 refers to cloth pressing roller shaft; 67 refers to short rod; 68 refers to rubber arc strip; 7 refers to unwinding control mechanism; 71 refers to T-shaped connecting rod; 72 refers to pressing vertical rod; 73 refers to stressed plate; 74 refers to vertically pressing connecting rod; 75 refers to fixing transverse plate; 76 refers to rolling arc plate; 77 refers to arc plate connecting rod; 78 refers to adaptive arc plate; 79 refers to bracket; 710 refers to cloth winding roller; 8 refers to vertical sliding rod; 9 refers to detection bottom plate; and 10 refers to slope plate.

### DETAILED DESCRIPTION OF PREFERRED EMBODIMENTS

The technical solutions in the embodiments of the present invention are clearly and completely described with reference to the drawings in the embodiments of the present invention. Apparently, the described embodiments are only some but not all of the embodiments of the present invention. Based on the embodiments in the present invention, all other embodiments obtained by those of ordinary skills in the art without going through any creative work should fall within the scope of protection of the present invention.

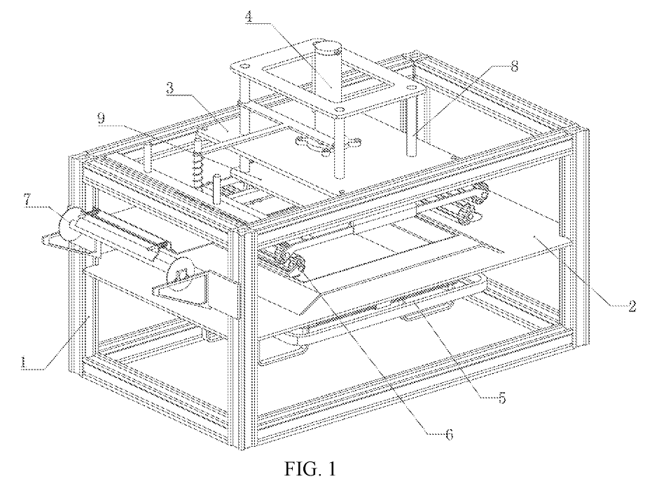

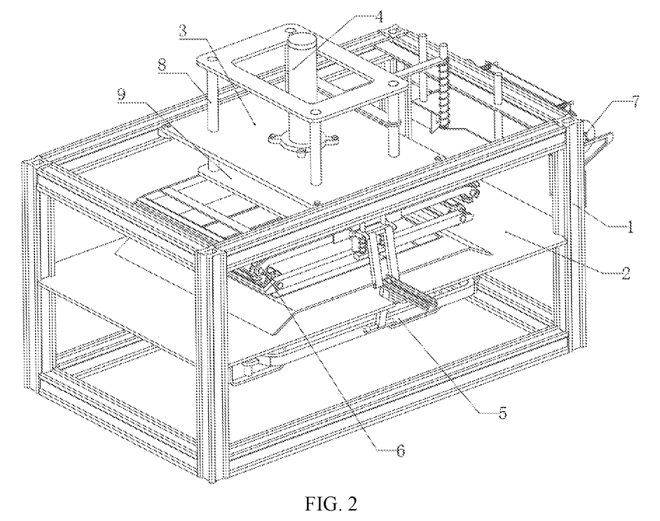

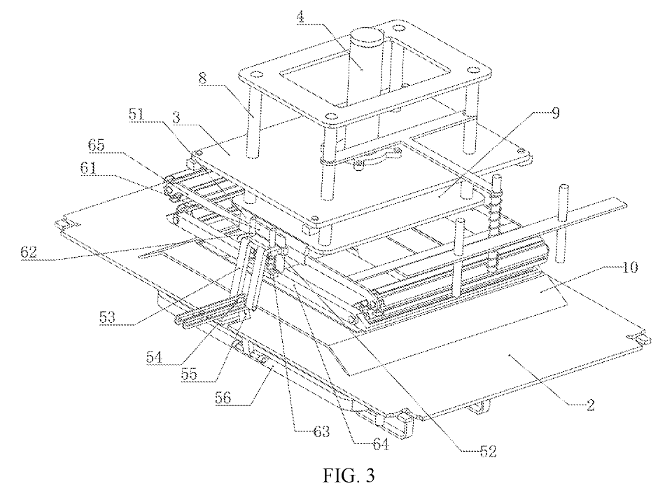

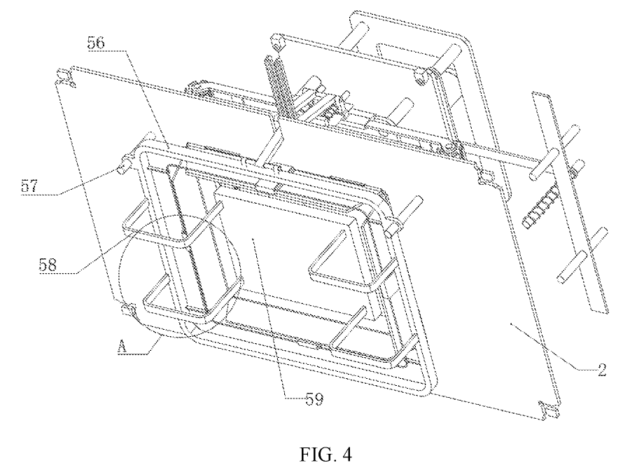

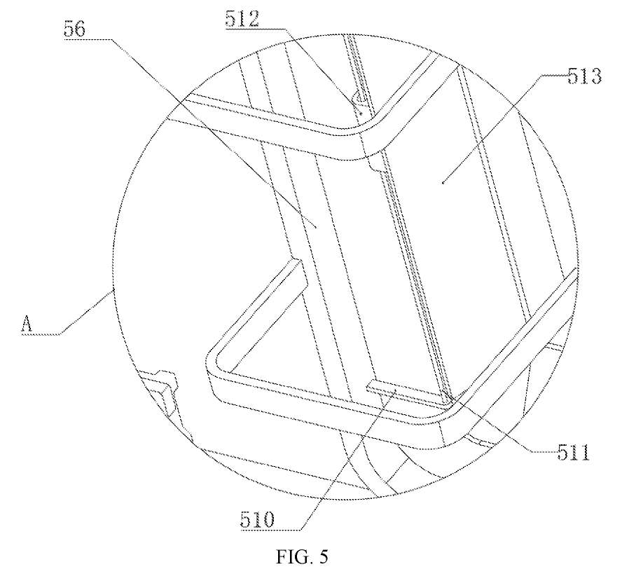

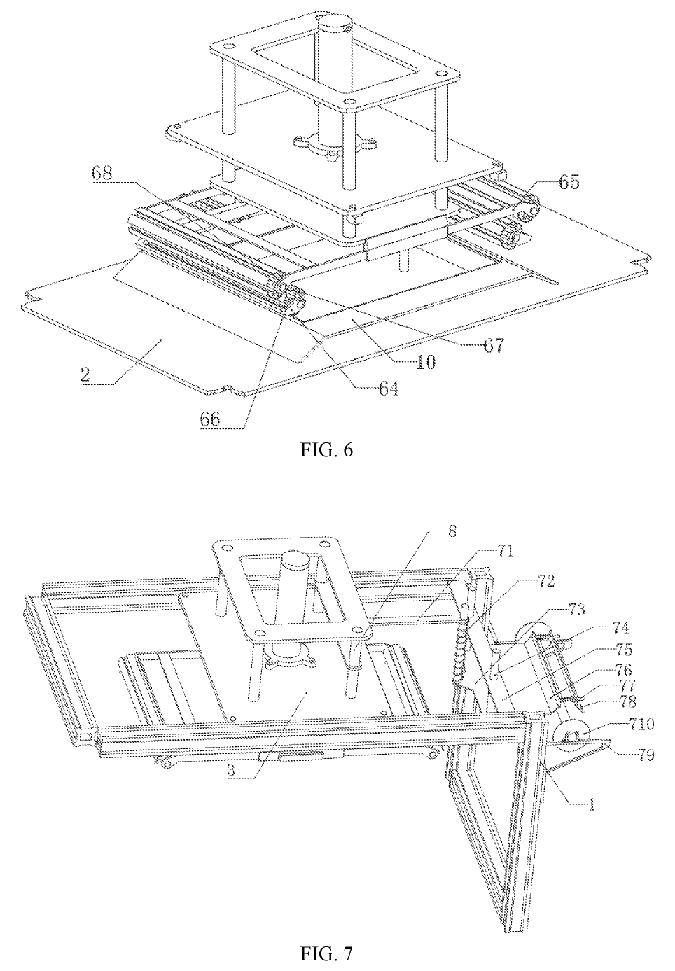

With reference to FIG. 1 to FIG. 7, the embodiment of the present invention is a fabric light transmittance testing device, which comprises a supporting frame 1, wherein a middle portion of an inner side of the supporting frame 1 is connected with a platform plate 2, a top surface of the supporting frame 1 is fixedly connected with a top fixing plate 3, a top surface of the top fixing plate 3 is fixedly connected with a hydraulic rod 4, a bottom surface of the platform plate 2 is fixedly connected with a light transmission testing mechanism 5, a cloth fastening mechanism 6 is arranged above the light transmission testing mechanism 5, one side of the top fixing plate 3 is provided with an unwinding control mechanism 7, four vertical sliding rods 8 slidably penetrate through an upper surface of the top fixing plate 3, a bottom surface of the vertical sliding rod 8 is fixedly connected with a detection bottom plate 9, and a top surface of the platform plate 2 is fixedly connected with a slope plate 10; the light transmission testing mechanism 5 comprises a side fixing plate 51, the side fixing plate 51 is fixedly connected to a rear side surface of the detection bottom plate 9, one side of the side fixing plate 51 far away from the detection bottom plate 9 is fixedly connected with a vertical-groove transverse plate 52, one side of the vertical-groove transverse plate 52 far away from the side fixing plate 51 is hinged with a double hinge rod 53, one end of the double hinge rod 53 far away from the vertical-groove transverse plate 52 is hinged with a single hinge rod 55, a hinged part between the double hinge rod 53 and the single hinge rod 55 is slidably connected with a guide groove rod 54, one end of the single hinge rod 55 far away from the double hinge rod 53 is connected with a lifting frame 56, two sides of the lifting frame 56 are both slidably connected with a frame guide rod 57, an inner side of the lifting frame 56 far away from the frame guide rod 57 is fixedly connected with a thin rod 510, one end of the thin rod 510 far away from the lifting frame 56 is fixedly connected with a guide frame rod 511, an outer side of the guide frame rod 511 is slidably connected with an inclined groove plate 512, a side surface of the inclined groove plate 512 is fixedly connected with a reflective plate 513, a bottom surface of the lifting frame 56 is fixedly connected with a bending rod 58, and one end of the bending rod 58 far away from the lifting frame 56 is fixedly connected with a light source plate 59; a bottom surface of the detection bottom plate 9 is provided with a photosensitive sensor, the guide groove rod 54 is fixedly connected with a rear side surface of the slope plate 10, the frame guide rod 57 is fixedly connected with a bottom surface of the platform plate 2, and the reflective plate 513 is hinged with the bottom surface of the platform plate 2. A light source emitted by the light source plate 59 passes through the cloth to reach the bottom surface of the detection bottom plate 9, and the photosensitive sensor on the bottom surface of the detection bottom plate 9 receives a light source signal and transmits the light source signal to a terminal. The terminal analyzes a light signal intensity to judge a light transmission effect of the cloth, so as to achieve the function of testing the light transmittance of the cloth. Meanwhile, the upward movement of the lifting frame 56 may drive the connected thin rod 510 and guide frame rod 511 to move upwardly, and the guide frame rod 511 moves upwardly and slides along the inclined groove plate 512, so that the inclined groove plate 512 drives the connected reflective plate 513 to deflect around a joint with the bottom surface of the platform plate 2, and the deflected reflective plate 513 reflects the light rays emitted by the light source plate 59, thereby improving the uniformity of light rays projected on the cloth, increasing a light irradiation range, and avoiding a structural shadow from blocking the light rays to affect the light transmittance of the cloth.

The cloth fastening mechanism 6 comprises a vertical sliding plate 61, the vertical sliding plate 61 is slidably connected to an upper surface of the vertical-groove transverse plate 52, the upper surface of the vertical-groove transverse plate 52 slidably penetrates through two elastic rods 62, a bottom surface of the elastic rod 62 is fixedly connected with an elastic rod fixing block 63, and one side of the elastic rod fixing block 63 oriented to the vertical sliding plate 61 is fixedly connected with lower roller frames 64. Two upper roller frames 65 are arranged above the lower roller frames 64, side surfaces of the upper roller frames 65 and the lower roller frames 64 are all rotatably connected with two cloth pressing roller shafts 66, a plurality of short rods 67 are distributed on an outer surface of the cloth pressing roller shaft 66 in an array, and one end of the short rod 67 far away from the cloth pressing roller shaft 66 is fixedly connected with a rubber arc strip 68. An outer side surface of the elastic rod 62 is provided with a spring, two ends of the spring are respectively fixedly connected with the vertical-groove transverse plate 52 and the elastic rod fixing block 63, the lower roller frame 64 is fixedly connected with the vertical sliding plate 61, the vertical sliding plate 61 penetrates from a top surface to a bottom surface of the vertical-groove transverse plate 52, and the upper roller frame 65 is slidably connected with the side fixing plate 51. A spring leaf is arranged inside the side fixing plate 51, two ends of the spring leaf are respectively fixedly connected with the side fixing plate 51 and the upper roller frame 65, the short rod 67 is slidably connected with the cloth pressing roller shaft 66, one end of the short rod 67 close to the cloth pressing roller shaft 66 is provided with a spring, and two ends of the spring are respectively fixedly connected with the cloth pressing roller shaft 66 and the short rod 67, so that the rubber arc strips 68 are pressed to push the short rods 67 to retract into the cloth pressing roller shafts 66 to squeeze the springs, and at this time, the pressed rubber arc strips 68 may contact with the cloth together with other rubber arc strips 68, thereby increasing a contact area of the cloth and fixing the cloth. In addition, the cloth pressing roller shaft 66 on the lower roller frame 64 may rotate around the cloth pressing roller shaft 66 on the lower roller frame 64, so that the lower roller frames 64 on two sides of the side fixing plate 51 pull the springs and move away from each other, thereby fastening the cloth contacting with the cloth pressing roller shaft 66, and preventing the cloth from being loose and wrinkled to affect the accuracy of light transmission testing.

The unwinding control mechanism 7 comprises a T-shaped connecting rod 71, the T-shaped connecting rod 71 is arranged on one side of the top fixing plate 3, one end of the T-shaped connecting rod 71 far away from the vertical sliding rod 8 is slidably connected with a pressing vertical rod 72, a bottom end of the pressing vertical rod 72 is fixedly connected with a stressed plate 73, a top surface of the stressed plate 73 is fixedly connected with two vertically pressing connecting rods 74, a top end of the vertically pressing connecting rod 74 is slidably connected with a fixing transverse plate 75, one side of the stressed plate 73 far away from the pressing vertical rod 72 is fixedly connected with a rolling arc plate 76, one end of the rolling arc plate 76 far away from the stressed plate 73 is fixedly connected with an arc plate connecting rod 77, one end of the arc plate connecting rod 77 far away from the rolling arc plate 76 is slidably connected with an adaptive arc plate 78, a cloth winding roller 710 is arranged below the adaptive arc plate 78, and a side surface of the cloth winding roller 710 is rotatably connected with a bracket 79. The T-shaped connecting rod 71 is fixedly connected with two vertical sliding rods 8, the outer side of the pressing vertical rod 72 is provided with the spring, and two ends of the spring are respectively fixedly connected with the T-shaped connecting rod 71 and the stressed plate 73. The fixing transverse plate 75 is fixedly connected with the supporting frame 1, the outer side of the arc plate connecting rod 77 is provided with the spring, and two ends of the spring are respectively fixedly connected with the rolling arc plate 76 and the adaptive arc plate 78. The supporting frame 1 is fixedly connected with the bracket 79, and the stressed plate 73 presses the cloth outside the cloth winding roller 710, thereby preventing the cloth wound outside the cloth winding roller 710 from being loose and offset to affect cloth detection. Meanwhile, with the unwinding of the cloth outside the cloth winding roller 710, a diameter of the cloth wound outside the cloth winding roller 710 will be gradually reduced, and at this time, the rolling arc plate 76 and the adaptive arc plate 78 covered above the cloth winding roller 710 may make the rolling arc plate 76 and the adaptive arc plate 78 approach each other under an elastic force of the spring outside the arc plate connecting rod 77, thereby improving a binding effect of the cloth outside the cloth winding roller 710, and improving the applicability to cloth rolls with different diameters outside the cloth winding roller 710.

Working principle: the hydraulic rod 4 extends downwardly to drive the detection bottom plate 9 to move downwardly, the detection bottom plate 9 drives the vertical-groove transverse plate 52 on a side surface of the side fixing plate 51 to move together, and a joint between the double hinge rod 53 and the single hinge rod 55 for connecting the vertical-groove transverse plate 52 slides along the guide groove rod 54. At this time, the lifting frame 56 connected with the single hinge rod 55 moves upwardly along the frame guide rod 57 to approach the bottom surface of the platform plate 2, and the bending rod 58 connected with the lifting frame 56 pushes the light source plate 59 to approach a bottom portion of cloth. A light source emitted by the light source plate 59 passes through the cloth to reach the bottom surface of the detection bottom plate 9, and the photosensitive sensor on the bottom surface of the detection bottom plate 9 receives a light source signal and transmits the light source signal to a terminal. The terminal analyzes a light signal intensity to judge a light transmission effect of the cloth, so as to achieve the function of testing the light transmittance of the cloth. Meanwhile, the upward movement of the lifting frame 56 may drive the connected thin rod 510 and guide frame rod 511 to move upwardly, and the guide frame rod 511 moves upwardly and slides along the inclined groove plate 512, so that the inclined groove plate 512 drives the connected reflective plate 513 to deflect around a joint with the bottom surface of the platform plate 2, and the deflected reflective plate 513 reflects the light rays emitted by the light source plate 59, thereby improving the uniformity of light rays projected on the cloth, increasing a light irradiation range, and avoiding a structural shadow from blocking the light rays to affect the light transmittance of the cloth.

Before the hydraulic rod 4 extends downwardly, there is a gap between the vertical-groove transverse plate 52 and the elastic rod fixing block 63 formed through an elastic force of the spring outside the elastic rod 62, there is a maximum distance between the lower roller frame 64 and the upper roller frame 65 at this time, and there is a gap between the cloth pressing roller shaft 66 of the lower roller frame 64 and the cloth pressing roller shaft 66 of the upper roller frame 65. At this time, the cloth is put in from the lower roller frame 64 on one side without blocking, and the cloth passes through a space between the cloth pressing roller shaft 66 of the lower roller frame 64 and the cloth pressing roller shaft 66 of the upper roller frame 65, which facilitates putting the cloth in a designated position before testing, and saves time for the cloth to pass through. When the hydraulic rod 4 starts to extend downwardly, the vertical-groove transverse plate 52 downwardly drives the elastic rod 62 and the elastic rod fixing block 63 to downwardly approach the slope plate 10, the lower roller frame 64 connected with the elastic rod fixing block 63 approaches the slope plate 10 to make the cloth pressing roller shaft 66 on the lower roller frame 64 contact with an inclined surface of the slope plate 10 through the adaptive arc plate 78, and the side fixing plate 51 continues to move downwardly at this time, so that the upper roller frame 65 connected with the side fixing plate 51 contacts with the cloth through the rubber arc strip 68 on the cloth pressing roller shaft 66 and squeezes the cloth pressing roller shaft 66 on the lower roller frame 64. The two cloth pressing roller shafts 66 are mutually contacted and pressed through the rubber arc strips 68, so that the rubber arc strips 68 are pressed to push the short rods 67 to retract into the cloth pressing roller shafts 66 to squeeze the springs, and at this time, the pressed rubber arc strips 68 may contact with the cloth together with other rubber arc strips 68, thereby increasing a contact area of the cloth and fixing the cloth. In addition, the cloth pressing roller shaft 66 on the lower roller frame 64 may rotate around the cloth pressing roller shaft 66 on the lower roller frame 64, so that the lower roller frames 64 on two sides of the side fixing plate 51 pull the springs and move away from each other, thereby fastening the cloth contacting with the cloth pressing roller shaft 66, and preventing the cloth from being loose and wrinkled to affect the accuracy of light transmission testing.

When the vertical sliding rod 8 moves along with the downward extension of the hydraulic rod 4, the movement of the vertical sliding rod 8 may drive the connected T-shaped connecting rod 71 to move, and the T-shaped connecting rod 71 moves downwardly to push the pressing vertical rod 72 and the stressed plate 73 to move. At this time, the rolling arc plate 76 and the adaptive arc plate 78 do not contact with the cloth outside the cloth winding roller 710, and when the T-shaped connecting rod 71 moves downwardly, the spring on the outer side the pressing vertical rod 72 is not pressed and moves downwardly together with the stressed plate 73. When the rolling arc plate 76 and the adaptive arc plate 78 contact with the cloth winding roller 710, the stressed plate 73 connected with the rolling arc plate 76 is subjected to a reverse action, so that the stressed plate 73 approaches the T-shaped connecting rod 71 and squeezes the spring on the outer side of the pressing vertical rod 72, and the cloth outside the cloth winding roller 710 is pressed through the stressed plate 73, which prevents the cloth wound outside the cloth winding roller 710 from being loose and offset to affect cloth detection. Meanwhile, with the unwinding of the cloth outside the cloth winding roller 710, a diameter of the cloth wound outside the cloth winding roller 710 will be gradually reduced, and at this time, the rolling arc plate 76 and the adaptive arc plate 78 covered above the cloth winding roller 710 may make the rolling arc plate 76 and the adaptive arc plate 78 approach each other under an elastic force of the spring outside the arc plate connecting rod 77, thereby improving a binding effect of the cloth outside the cloth winding roller 710, and improving the applicability to cloth rolls with different diameters outside the cloth winding roller 710.

It should be noted that relational terms herein, such as first, second, and the like, are used merely to distinguish one entity or operation from another entity or operation, and do not necessarily require or imply that there is any such relationship or order between these entities or operations. Furthermore, the terms "comprise", "include", or any other variation thereof, are intended to cover a non-exclusive inclusion, so that a process, a method, an article, or equipment that comprises a list of elements not only includes those elements but also includes other elements not expressly listed, or further includes elements inherent to such process, method, article, or equipment.

Finally, it should be noted that: the above is only the preferred embodiments of the present invention, and the preferred embodiments are not intended to limit the present invention. Although the present invention is described in detail with reference to the above embodiments, those skilled in the art may still modify the technical solutions recorded in the above embodiments, or make equivalent replacements to some of the technical features. Any modification, equivalent substitution, improvement, etc. made within the spirit and principle of the present invention are included in the scope of protection of the present invention.

We claim:

1. A fabric light transmittance testing device, comprising a supporting frame (1), wherein a middle portion of an inner side of the supporting frame (1) is connected with a platform plate (2), a top surface of the supporting frame (1) is fixedly connected with a top fixing plate (3), a top surface of the top fixing plate (3) is fixedly connected with a hydraulic rod (4), a bottom surface of the platform plate (2) is fixedly connected with a light transmission testing mechanism (5), a cloth fastening mechanism (6) is arranged above the light transmission testing mechanism (5), one side of the top fixing plate (3) is provided with an unwinding control mechanism (7), four vertical sliding rods (8) slidably penetrate through an upper surface of the top fixing plate (3), a bottom surface of the vertical sliding rod (8) is fixedly connected with a detection bottom plate (9), and a top surface of the platform plate (2) is fixedly connected with a slope plate (10);
the light transmission testing mechanism (5) comprises a side fixing plate (51), the side fixing plate (51) is fixedly connected to a rear side surface of the detection bottom plate (9), one side of the side fixing plate (51) far away from the detection bottom plate (9) is fixedly connected with a vertical-groove transverse plate (52), one side of the vertical-groove transverse plate (52) far away from the side fixing plate (51) is hinged with a double hinge rod (53), one end of the double hinge rod (53) far away from the vertical-groove transverse plate (52) is hinged with a single hinge rod (55), a hinged part between the double hinge rod (53) and the single hinge rod (55) is slidably connected with a guide groove rod (54), one end of the single hinge rod (55) far away from the double hinge rod (53) is connected with a lifting frame (56), two sides of the lifting frame (56) are both slidably connected with a frame guide rod (57), an inner side of the lifting frame (56) far away from the frame guide rod (57) is fixedly connected with a thin rod (510), one end of the thin rod (510) far away from the lifting frame (56) is fixedly connected with a guide frame rod (511), an outer side of the guide frame rod (511) is slidably connected with an inclined groove plate (512), a side surface of the inclined groove plate (512) is fixedly connected with a reflective plate (513), a bottom surface of the lifting frame (56) is fixedly connected with a bending rod (58), and one end of the bending rod (58) far away from the lifting frame (56) is fixedly connected with a light source plate (59);
a bottom surface of the detection bottom plate (9) is provided with a photosensitive sensor, the guide groove rod (54) is fixedly connected with a rear side surface of the slope plate (10), the frame guide rod (57) is fixedly connected with a bottom surface of the platform plate (2), and the reflective plate (513) is hinged with the bottom surface of the platform plate (2); and
the cloth fastening mechanism (6) comprises a vertical sliding plate (61), the vertical sliding plate (61) is slidably connected to an upper surface of the vertical-groove transverse plate (52), the upper surface of the vertical-groove transverse plate (52) slidably penetrates through two elastic rods (62), a bottom surface of the elastic rod (62) is fixedly connected with an elastic rod fixing block (63), and one side of the elastic rod fixing block (63) oriented to the vertical sliding plate (61) is fixedly connected with a lower roller frame (64).

2. The fabric light transmittance testing device according to claim 1, wherein two upper roller frames (65) are arranged above the lower roller frames (64), side surfaces of the upper roller frames (65) and the lower roller frames (64) are all rotatably connected with two cloth pressing roller shafts (66), a plurality of short rods (67) are distributed on an outer surface of the cloth pressing roller shaft (66) in an array, and one end of the short rod (67) far away from the cloth pressing roller shaft (66) is fixedly connected with a rubber arc strip (68).

3. The fabric light transmittance testing device according to claim 2, wherein an outer side surface of the elastic rod (62) is provided with a spring, two ends of the spring are respectively fixedly connected with the vertical-groove transverse plate (52) and the elastic rod fixing block (63), the lower roller frame (64) is fixedly connected with the vertical sliding plate (61), the vertical sliding plate (61) penetrates from a top surface to a bottom surface of the vertical-groove transverse plate (52), and the upper roller frame (65) is slidably connected with the side fixing plate (51).

4. The fabric light transmittance testing device according to claim 3, wherein a spring leaf is arranged inside the side fixing plate (51), two ends of the spring leaf are respectively fixedly connected with the side fixing plate (51) and the upper roller frame (65), the short rod (67) is slidably connected with the cloth pressing roller shaft (66), one end of the short rod (67) close to the cloth pressing roller shaft (66) is provided with a spring, and two ends of the spring are respectively fixedly connected with the cloth pressing roller shaft (66) and the short rod (67).

5. The fabric light transmittance testing device according to claim 4, wherein the unwinding control mechanism (7) comprises a T-shaped connecting rod (71), the T-shaped connecting rod (71) is arranged on one side of the top fixing plate (3), one end of the T-shaped connecting rod (71) far away from the vertical sliding rod (8) is slidably connected with a pressing vertical rod (72), a bottom end of the pressing vertical rod (72) is fixedly connected with a stressed plate (73), a top surface of the stressed plate (73) is fixedly connected with two vertically pressing connecting rods (74), a top end of the vertically pressing connecting rod (74) is slidably connected with a fixing transverse plate (75), one side of the stressed plate (73) far away from the pressing vertical rod (72) is fixedly connected with a rolling arc plate (76), one end of the rolling arc plate (76) far away from the stressed plate (73) is fixedly connected with an arc plate connecting rod (77), one end of the arc plate connecting rod (77) far away from the rolling arc plate (76) is slidably connected with an adaptive arc plate (78), a cloth winding roller (710) is arranged below the adaptive arc plate (78), and a side surface of the cloth winding roller (710) is rotatably connected with a bracket (79).

6. The fabric light transmittance testing device according to claim 5, wherein the T-shaped connecting rod (71) is fixedly connected with two vertical sliding rods (8), an outer side of the pressing vertical rod (72) is provided with a spring, two ends of the spring are respectively fixedly connected with the T-shaped connecting rod (71) and the stressed plate (73), and the fixing transverse plate (75) is fixedly connected with the supporting frame (1).

7. The fabric light transmittance testing device according to claim 6, wherein an outer side of the arc plate connecting rod (77) is provided with a spring, two ends of the spring are respectively fixedly connected with the rolling arc plate (76) and the adaptive arc plate (78), and the supporting frame (1) is fixedly connected with the bracket (79).
